(19) United States
(12) Patent Application Publication
(10) Pub. No.: US 2005/0113410 A1
(43) Pub. Date: May 26, 2005

(54) Pharmaceutical salts of zafirlukast

(76) Inventors: Mark Tawa, West Roxbury, MA (US); Orn Almarsson, Shrewsbury, MA (US); Julius Remenar, Framingham, MA (US)

Correspondence Address: TRANSFORM PHARMACEUTICALS, INC., 29 HARTWELL AVENUE, LEXINGTON, MA, 02421, US

(21) Appl. No.: 10/975,915

(22) Filed: Oct. 28, 2004

Related U.S. Application Data
(60) Provisional application No. 60/516,797, filed on Nov. 3, 2003.

(51) Int. Cl. A61K 31/47

(52) U.S. Cl. 514/311

(57) ABSTRACT

Zafirlukast, a leukotriene receptor antagonist, converts to a less bioavailable form in the presence of water. The present invention relates to crystalline forms of zafirlukast including salts and solvates of zafirlukast substantially more stable in water than presently marketed zafirlukast. A method of preparing the compounds of the present invention is disclosed, as well as a method of treating asthma.

### CROSS-REFERENCE TO RELATED APPLICATION

The present application claims the benefit of priority of U.S. Provisional Application Ser. No. 60/516,797, filed Nov. 3, 2003, which is hereby incorporated by reference herein in its entirety, including any figures, tables or drawings.

### BACKGROUND OF THE INVENTION

Zafirlukast, 4-(5-cyclopentyloxy-carbonylamino-1-methyl-indol-3-ylmethyl)-3-methoxy-N-o-tolylsulfonylbenzamide, is represented by the structural formula (I):

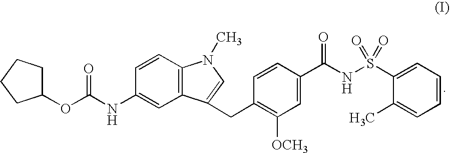

Zafirlukast belongs to the general class of leukotriene receptor antagonists. Cysteinyl leukotriene production and receptor occupation have been correlated with the pathophysiology of asthma. The synthesis and use of zafirlukast are further described in U.S. Pat. Nos. 4,859,692, 5,294,636, 5,319,097, 5,482,963, 5,583,152, 5,612,367 6,143,775, 6,333,361, and 6,399,104, the contents of which are incorporated herein by reference in their entireties.

In its commercially available form as ACCOLATE®, zafirlukast is a neutral molecule that is essentially insoluble in water. It is desirable in the treatment of a number of diseases, both therapeutically and prophylactically, to provide the active pharmaceutical ingredient (API) in a form that provides a modified release profile. Such modified release profiles may, in certain circumstances, include controlled release, extended release, or sustained release profiles. The modified release formulation provides an alternative dosage form and/or regime which adds to the physician's armory. Preferably the modified release provides a generally uniform and constant rate of release over an extended period of time which achieves a stable and desired blood (plasma) level of the active ingredient preferably without the need for frequent administration of the medicament.

Before a compound in the solid state can be formulated in a pharmaceutical composition, a physical form of the compound is sought which is physically stable and can be prepared substantially free of other physical forms. This latter requirement is important because different physical forms can have markedly different bioavailabilities.

### SUMMARY OF THE INVENTION

Amorphous neutral zafirlukast is known to convert to a monohydrate form in the presence of water. The monohydrate has a decreased bioavailability from that of the amorphous form. It has now been found that a crystalline salt of zafirlukast can be isolated following reaction of neutral zafirlukast with strong base. This crystalline salt of zafirlukast has additionally been found to be particularly stable in water. The salt of zafirlukast is stable under both acidic and neutral aqueous conditions.

The present invention includes a crystalline salt of zafirlukast. In particular, the invention includes a composition comprising a crystalline alkali metal salt of zafirlukast. Such salts can be crystallized with a second crystalline entity, where the two entities may form a co-crystal. Types of crystals include polymorphs, solvates, desolvates, hydrates, dehydrates, anhydrous forms, and co-crystals thereof. Compositions of the present invention are advantageously substantially more stable in water than presently marketed zafirlukast. A pharmaceutical composition comprises a crystalline salt of zafirlukast described herein, in combination with one or more pharmaceutically acceptable carriers or diluents.

In one embodiment, the present invention is a crystalline salt of zafirlukast and a method of preparing said crystalline salt of zafirlukast. The method comprises:
a. contacting zafirlukast with a solvent and a base;
b. reacting zafirlukast with at least one equivalent of one or more bases; and
c. isolating said crystalline salt, thereby obtaining crystals of said salt of zafirlukast.
   The solvent and the base can be combined before contacting with zafirlukast, or the zafirlukast can be dissolved in the solvent followed by the addition of base.

In another embodiment, the present invention is a potassium salt of zafirlukast, wherein the salt is characterized by a powder X-ray diffraction pattern having peaks, for example, at 2-theta angles of 5.37, 7.77 and 17.05 degrees or a diffraction pattern substantially the same as in FIG. 3.

The invention also includes a method of treating a subject suffering from asthma comprising administering to said subject one or more compositions of the present invention, where the composition produces a therapeutic effect. Preferably, the composition is administered orally.

One embodiment of the present invention is a method of preparing a salt of zafirlukast. Another embodiment includes the preparation of a pharmaceutically acceptable form of zafirlukast having increased bioavailability over the monohydrate, the nonsolvated crystal, and the amorphous neutral forms of the API. Another embodiment includes a pharmaceutically acceptable form of zafirlukast having increased bioavailability over the monohydrate, the nonsolvated crystal, or the amorphous neutral forms of the API. The present invention also provides a form of zafirlukast that is more stable in the presence of water than the amorphous neutral form.

### BRIEF DESCRIPTION OF THE DRAWINGS

FIG. 1 shows the thermogravimetric analysis (TGA) thermogram of zafirlukast potassium salt.

FIG. 2 shows the differential scanning calorimetry (DSC) thermogram of zafirlukast potassium salt.

FIG. 3 shows the powder x-ray diffractogram (PXRD) of zafirlukast potassium salt.

FIG. 4 shows the thermogravimetric analysis (TGA) thermogram of zafirlukast methanol solvate.

FIG. 5 shows the differential scanning calorimetry (DSC) of zafirlukast methanol solvate.

FIG. 6 shows the powder x-ray diffractogram (PXRD) of zafirlukast methanol solvate.

FIG. 7 shows the Raman spectrum of zafirlukast methanol solvate.

### DETAILED DESCRIPTION OF THE INVENTION

The present invention relates to crystalline forms of zafirlukast including crystal solvates and salts of zafirlukast, which are significantly more stable in water than presently marketed amorphous zafirlukast. For purposes of the present invention, “neutral zafirlukast” refers to zafirlukast that is uncharged, such as the presently marketed form of zafirlukast, which is known by the tradename ACCOLATE®. For ease of reference the term “zafirlukast” when used alone means either neutral zafirlukast or a salt thereof unless specified as neutral zafirlukast or a salt of zafirlukast.

The term “co-crystal” as used herein means a crystalline material comprised of two or more unique solids at room temperature, each containing distinctive physical characteristics, such as structure, melting point and heats of fusion, with the exception that, if specifically stated, the API may be a liquid at room temperature. The co-crystals of the present invention comprise a co-crystal former H-bonded to an API. The co-crystal former may be H-bonded directly to the API or may be H-bonded to an additional molecule which is bound to the API. The additional molecule may be H-bonded to the API or bound ionically or covalently to the API. The additional molecule could also be a different API. Solvates of API compounds that do not further comprise a co-crystal forming compound are not co-crystals according to the present invention. The co-crystals may however, include one or more solvent molecules in the crystalline lattice. That is, solvates of co-crystals, or a co-crystal further comprising a solvent or compound that is a liquid at room temperature, is included in the present invention, but crystalline material comprised of only one solid and one or more liquids (at room temperature) are not included in the present invention. The co-crystals may also be a co-crystal between a co-crystal former and a salt of an API, but the API and the co-crystal former of the present invention are constructed or bonded together through hydrogen bonds. Other modes of molecular recognition may also be present including, pi-stacking, guest-host complexation and van der Waals interactions. Of the interactions listed above, hydrogen-bonding is the dominant interaction in the formation of the co-crystal, (and a required interaction according to the present invention) whereby a non-covalent bond is formed between a hydrogen bond donor of one of the moieties and a hydrogen bond acceptor of the other. An alternative embodiment provides for a co-crystal wherein the co-crystal former is a second API. In another embodiment, the co-crystal former is not an API. In another embodiment the co-crystal comprises two co-crystal formers. Co-crystals may also be formed where the API is a “guest” molecule in regions of a crystalline lattice formed by the co-crystal forming compound, thus forming an inclusion complex.

The term “solvate” as used herein is defined as a solid compound formed by solvation, for example as a combination of solvent molecules with molecules or ions of a solute. Well known solvent molecules include water, alcohols and other polar organic solvents. Alcohols include methanol, ethanol, n-propanol, isopropanol, n-butanol, isobutanol, and t-butanol. Alcohols also include polymerized alcohols such as polyalkylene glycols (e.g., polyethylene glycol, polypropylene glycol). The best-known and preferred solvent is typically water, and solvate compounds formed by solvation with water are termed hydrates. In one embodiment, the solvates are crystalline.

Solvates and co-crystals of zafirlukast can be prepared by crystallizing zafirlukast from an organic solvent in the presence of an organic molecule that is capable of donating and/or accepting a hydrogen bonding interaction to zafirlukast. The organic solvent(s) could be the molecule that is to become the solvate. The formation of co-crystal solvates can be achieved in solutions where both an API and a co-crystal former are dissolved. The solvate or co-crystal will not form unless the solvate or co-crystal forming molecule has favorable intermolecular interactions with zafirlukast. Typical solvate or co-crystal forming molecules include water (hydrates), alcohols (e.g., methanol, ethanol, propanol, isopropanol, butanol, isobutanol, tert-butanol, amyl alcohol, isoamyl alcohol), amides, amines, and carboxylic acids.

Salts of zafirlukast are formed by reaction of zafirlukast with an acceptable base. Acceptable bases include, but are not limited to, metal hydroxides and alkoxides. Metals include alkali metals (sodium, potassium, lithium, cesium), alkaline earth metals (magnesium, calcium), zinc, aluminum, and bismuth. Alkoxides include methoxide, ethoxide, n-propoxide, and isopropoxide. Additional bases include arginine, procaine, and other molecules having amino or guanidinium moieties with sufficiently high pKₐ's. Potassium hydroxide and potassium tert-butoxide are preferred bases. The amount of base used to form a salt is typically about one or more, about two or more, about three or more, about four or more, about five or more, or about ten or more equivalents relative to zafirlukast. In one embodiment, about one to about two equivalents of one or more bases are reacted with zafirlukast to form a salt.

A zafirlukast salt can be transformed into a second zafirlukast salt by transmetallation or another process that replaces the cation of the first zafirlukast salt. In one example, a potassium salt of zafirlukast is prepared and is subsequently reacted with a second salt such as an alkaline earth metal halide (e.g., MgBr₂, MgCl₂, CaCl₂, CaBr₂), an alkaline earth metal sulfate or nitrate (e.g., Mg(NO₃)₂, Mg(SO₄)₂, Ca(NO₃)₂, Ca(SO₄)₂), or an alkaline earth metal salt of an organic acid (e.g. calcium formate, magnesium formate, calcium acetate, magnesium acetate, calcium propionate, magnesium propionate) to form an alkaline earth metal salt of zafirlukast.

In another embodiment of the present invention, zafirlukast salts are substantially pure. A salt that is substantially pure can be greater than about 80% pure, greater than about 85% pure, greater than about 90% pure, greater than about 95% pure, greater than about 98% pure, or greater than about 99% pure. Purity of a salt can be measured with respect to the amount of salt (as opposed to unreacted neutral zafirlukast or base) or can be measured with respect to a specific polymorph, co-crystal, solvate, desolvate, hydrate, dehydrate, or anhydrous form of a salt.

A zafirlukast salt of the present invention is generally significantly more stable in water than presently marketed amorphous neutral zafirlukast, and is less hydrophobic than the amorphous neutral form. For example, the conversion of amorphous neutral zafirlukast to the crystalline monohydrate can occur 2 times, 3 times, 4 times, 5 times, 10 times, 25 times, 50 times, 100 times, 250 times, 500 times, 1000 times, 2500 times, 5000 times, or 10,000 times faster than the conversion of a zafirlukast crystalline form of the present invention to a neutral form.

A zafirlukast salt and a zafirlukast solvate or co-crystal of the present invention can be characterized by differential scanning calorimetry (DSC). The potassium salt of zafirlukast prepared in Example 1 is characterized by an endothermic transition observed by differential scanning calorimetry at about 258 degrees C. The methanol solvate prepared in Example 2 is characterized by an endothermic transition observed by differential scanning calorimetry at about 141 degrees C.

The zafirlukast salt and the zafirlukast solvate of the present invention can also be characterized by thermogravimetric analysis (TGA). The potassium salt of zafirlukast prepared by Example 1 was characterized by TGA, and the salt loses about 2 percent to about 5 percent of its weight when the temperature is raised from room temperature (about 25 degrees C.) to about 225 degrees C. The methanol solvate prepared by Example 2 loses about 5.3 percent of its weight between about 75 degrees C. and about 160 degrees C.

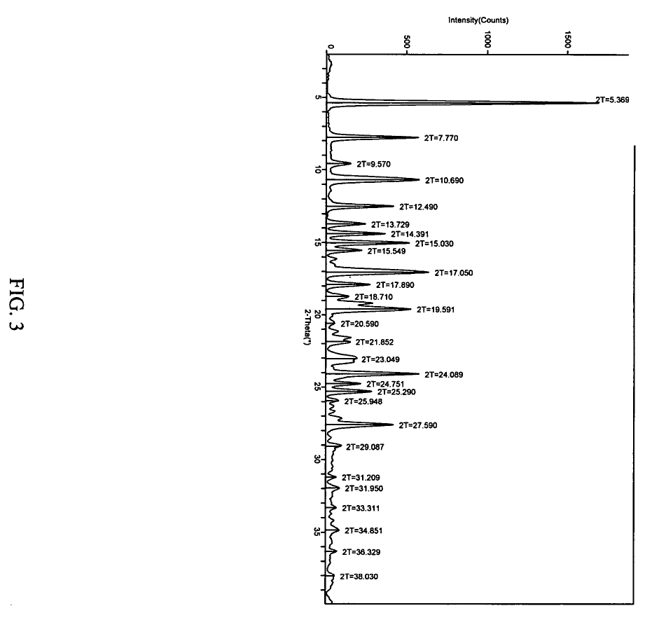

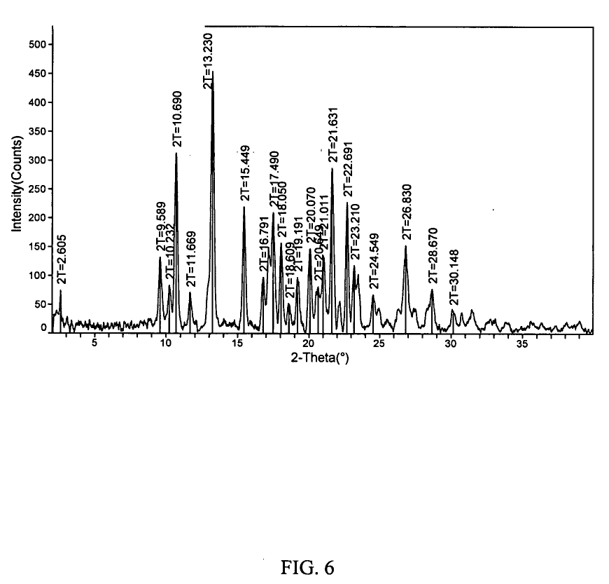

The zafirlukast salt and the zafirlukast solvate of the present invention can further be characterized by powder x-ray diffraction (PXRD). The potassium salt of zafirlukast prepared by Example 1 has peaks at 2-theta angles of 5.37, 7.77, 10.69, 12.49, 13.73, 15.03, 17.05, 19.59, 24.09, and 27.59 degrees. Any combination of one, two, three, four five, six, or more of the above peaks or any in FIG. 3 are characteristic of zafirlukast potassium salt. The methanol solvate of zafirlukast prepared by Example 2 has peaks at 2-theta angles of 9.59, 10.69, 13.23, 15.45, 17.49, 18.05, 21.63, 22.69, and 26.83 degrees. Any combination of one, two, three, four, five, six, or more of the above peaks or any in FIG. 6 are characteristic of zafirlukast methanol solvate.

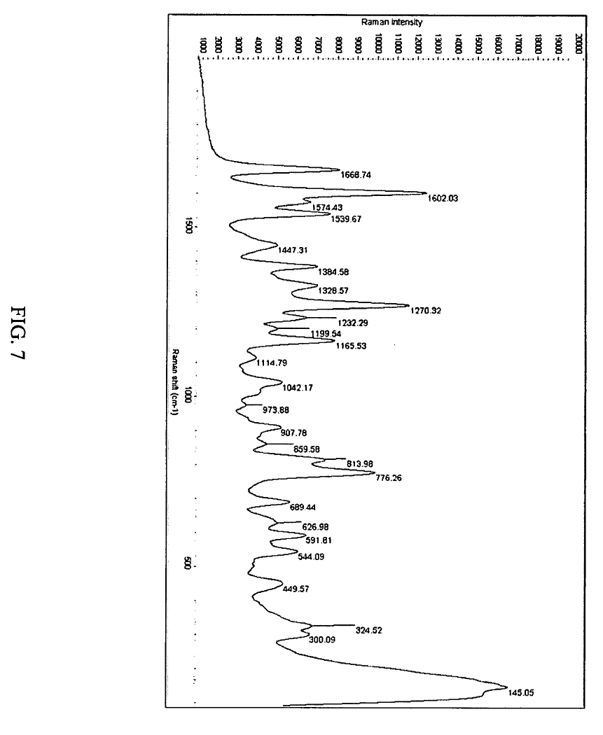

Raman spectroscopy was also used to characterize the zafirlukast methanol solvate of the present invention. When analyzed by Raman spectroscopy, the zafirlukast methanol solvate synthesized in Example 2 exhibited Raman shifts at 1669, 1602, 1540, 1385, 1270, 1166, 776, and 592 cm⁻¹. Any combination of one, two, three, four, or more of the above Raman shifts or any in FIG. 7 are characteristic of zafirlukast methanol solvate.

Another technique used to characterize the zafirlukast potassium salt of the present invention was elemental analysis. When analyzed by elemental analysis, the zafirlukast potassium salt was found to contain 60.59 percent C, 5.30 percent H, 6.77 percent N, and 6.35 percent K. This is in agreement with the calculated values of 60.66 percent C, 5.26 percent H, 6.85 percent N, and 6.37 percent K.

Zafirlukast salts can comprise solvate molecules and can occur in a variety of solvation states, also known as solvates. Different solvates of a zafirlukast salt can be obtained by varying the method of preparation. Solvates typically have different solubilities, such that a more thermodynamically stable solvate is less soluble than a less thermodynamically stable solvate. Solvates can also differ in properties such as shelf-life, bioavailability, morphology, vapor pressure, density, color, and shock sensitivity. In another embodiment, the shelf life of a zafirlukast salt of the present invention is at least one day, at least one week, at least two weeks, at least one month, at least three months, at least six months, at least one year, at least two years or at least five years.

Suitable solvate molecules include water, alcohols, other polar organic solvents, and combinations thereof. Alcohols include methanol, ethanol, n-propanol, isopropanol, n-butanol, isobutanol, and t-butanol. Water is a preferred solvent. Solvate molecules can be removed from a crystalline salt, such that the salt is either a partial or complete desolvate. If the solvate molecule is water (forming a hydrate), then a desolvated salt is said to be a dehydrate. A salt with all water removed is anhydrous. Solvate molecules can be removed from a salt by methods such as heating, treating under vacuum or reduced pressure, blowing air over a salt, or a combination thereof.

A zafirlukast salt of the present invention, in one of the above-listed forms, can co-crystallize with one or more other substances. The other substance or substances can be, for example, a salt, a free acid, or a free base, and can interact with a zafirlukast salt through hydrogen bonds and other energetically-favorable means.

Zafirlukast salts of the present invention are prepared by contacting zafirlukast with a solvent. Suitable solvents include water, alcohols, other polar organic solvents, and combinations thereof. Methanol is a preferred solvent. Zafirlukast is reacted with a base, where suitable bases are listed above, such that zafirlukast forms a salt and preferably dissolves. Bases can be added to zafirlukast with the solvent (i.e., dissolved in the solvent), such that zafirlukast is solvated and deprotonated essentially simultaneously, or bases can be added after the zafirlukast has been contacted with solvent. In the latter scenario, bases can either be dissolved in a solvent, which can be either the solvent already contacting zafirlukast or a different solvent can be added as a neat solid or liquid, or a combination thereof. Potassium hydroxide and potassium tert-butoxide are preferred bases. The amount of base required is discussed above. Evaporation of solvent, which yields an oil, can be followed by re-dissolving the salt in a suitable solvent for crystallization. Also, filtration followed by the addition of a seed crystal can be used as an alternate procedure to crystallize the zafirlukast salt. In each case, the suitable solvent or the seed crystal acts as a crystallization promoter for the salt. Depending on the solvent utilized, a zafirlukast salt may precipitate and/or crystallize independently of evaporation. Crystals of a zafirlukast salt can be filtered to remove bulk solvent. Methods of removing solvate molecules are discussed above.

Excipients employed in pharmaceutical compositions of the present invention can be solids, semi-solids, liquids or combinations thereof. Preferably, excipients are solids. Compositions of the invention containing excipients can be prepared by any known technique of pharmacy that comprises admixing an excipient with a drug or therapeutic agent. A pharmaceutical composition of the invention contains a desired amount of zafirlukast (or a salt or solvate thereof) per dose unit and, if intended for oral administration, can be in the form, for example, of a tablet, a caplet, a pill, a hard or soft capsule, a lozenge, a cachet, a dispensable powder, granules, a suspension, an elixir, a liquid, or any other form reasonably adapted for such administration. If intended for parenteral administration, it can be in the form, for example, of a suspension or transdermal patch. If intended for rectal administration, it can be in the form, for example, of a suppository. Presently preferred are oral dosage forms that are discrete dose units each containing a predetermined amount of the drug, such as tablets or capsules.

Non-limiting examples follow of excipients that can be used to prepare pharmaceutical compositions of the invention.

Pharmaceutical compositions of the invention optionally comprise one or more pharmaceutically acceptable carriers or diluents as excipients. Suitable carriers or diluents illustratively include, but are not limited to, either individually or in combination, lactose, including anhydrous lactose and lactose monohydrate; starches, including directly compressible starch and hydrolyzed starches (e.g., Celutab and Emdex); mannitol; sorbitol; xylitol; dextrose (e.g., Cerelose 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of alpha- and amorphous cellulose (e.g., Rexcel) and powdered cellulose; calcium carbonate; glycine; bentonite; polyvinylpyrrolidone; and the like. Such carriers or diluents, if present, constitute in total about 5% to about 99%, preferably about 10% to about 85%, and more preferably about 20% to about 80%, of the total weight of the composition. The carrier, carriers, diluent, or diluents selected preferably exhibit suitable flow properties and, where tablets are desired, compressibility.

Lactose, mannitol, dibasic sodium phosphate, and microcrystalline cellulose (particularly Avicel PH microcrystalline cellulose such as Avicel PH 101), either individually or in combination, are preferred diluents. These diluents are chemically compatible with zafirlukast. The use of extragranular microcrystalline cellulose (that is, microcrystalline cellulose added to a granulated composition) can be used to improve hardness (for tablets) and/or disintegration time. Lactose, especially lactose monohydrate, is particularly preferred. Lactose typically provides compositions having suitable release rates of zafirlukast, stability, pre-compression flowability, and/or drying properties at a relatively low diluent cost. It provides a high density substrate that aids densification during granulation (where wet granulation is employed) and therefore improves blend flow properties and tablet properties.

Pharmaceutical compositions of the invention optionally comprise one or more pharmaceutically acceptable disintegrants as excipients, particularly for tablet formulations. Suitable disintegrants include, but are not limited to, either individually or in combination, starches, including sodium starch glycolate (e.g., Explotab of PenWest) and pregelatinized corn starches (e.g., National 1551 of National Starch and Chemical Company, National 1550, and Colorcon 1500), clays (e.g., Veegum H V of R. T. Vanderbilt), celluloses such as purified cellulose, microcrystalline cellulose, methylcellulose, carboxymethylcellulose and sodium carboxymethylcellulose, croscarmellose sodium (e.g., Ac-Di-Sol of FMC), alginates, crospovidone, and gums such as agar, guar, locust bean, karaya, pectin and tragacanth gums.

Disintegrants may be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition.

Croscarmellose sodium is a preferred disintegrant for tablet or capsule disintegration, and, if present, preferably constitutes about 0.2% to about 10%, more preferably about 0.2% to about 7%, and still more preferably about 0.2% to about 5%, of the total weight of the composition. Croscarmellose sodium confers superior intragranular disintegration capabilities to granulated pharmaceutical compositions of the present invention.

Pharmaceutical compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, but are not limited to, either individually or in combination, acacia; tragacanth; sucrose; gelatin; glucose; starches such as, but not limited to, pregelatinized starches (e.g., National 1511 and National 1500); celluloses such as, but not limited to, methylcellulose and carmellose sodium (e.g., Tylose); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel of Aqualon); and ethylcellulose (e.g., Ethocel of the Dow Chemical Company). Such binding agents and/or adhesives, if present, constitute in total about 0.5% to about 25%, preferably about 0.75% to about 15%, and more preferably about 1% to about 10%, of the total weight of the pharmaceutical composition.

Pharmaceutical compositions of the invention optionally comprise one or more pharmaceutically acceptable wetting agents as excipients. Such wetting agents are preferably selected to maintain the zafirlukast in close association with water, a condition that is believed to improve bioavailability of the composition.

Non-limiting examples of surfactants that can be used as wetting agents in pharmaceutical compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween 80 of ICI), propylene glycol fatty acid esters, for example propylene glycol laurate (e.g., Lauroglycol of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the pharmaceutical composition.

Wetting agents that are anionic surfactants are preferred. Sodium lauryl sulfate is a particularly preferred wetting agent. Sodium lauryl sulfate, if present, constitutes about 0.25% to about 7%, more preferably about 0.4% to about 4%, and still more preferably about 0.5% to about 2%, of the total weight of the pharmaceutical composition.

Pharmaceutical compositions of the invention optionally comprise one or more pharmaceutically acceptable lubricants (including anti-adherents and/or glidants) as excipients. Suitable lubricants include, but are not limited to, either individually or in combination, glyceryl behapate (e.g., Compritol 888 of Gattefosse); stearic acid and salts thereof, including magnesium, calcium and sodium stearates; hydrogenated vegetable oils (e.g., Sterotex of Abitec); colloidal silica; talc; waxes; boric acid; sodium benzoate; sodium acetate; sodium fumarate; sodium chloride; DL-leucine; PEG (e.g., Carbowax 4000 and Carbowax 6000 of the Dow Chemical Company); sodium oleate; sodium lauryl sulfate; and magnesium lauryl sulfate. Such lubricants, if present, constitute in total about 0.1% to about 10%, preferably about 0.2% to about 8%, and more preferably about 0.25% to about 5%, of the total weight of the pharmaceutical composition.

Magnesium stearate is a preferred lubricant used, for example, to reduce friction between the equipment and granulated mixture during compression of tablet formulations.

Suitable anti-adherents include, but are not limited to, talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0.1% to about 10%, more preferably about 0.25% to about 5%, and still more preferably about 0.5% to about 2%, of the total weight of the pharmaceutical composition.

Glidants can be used to promote powder flow of a solid formulation. Suitable glidants include, but are not limited to, colloidal silicon dioxide, starch, talc, tribasic calcium phosphate, powdered cellulose and magnesium trisilicate. Colloidal silicon dioxide is particularly preferred.

Other excipients such as colorants, flavors and sweeteners are known in the pharmaceutical art and can be used in pharmaceutical compositions of the present invention. Tablets can be coated, for example with an enteric coating, or uncoated. Pharmaceutical compositions of the invention can further comprise, for example, buffering agents.

Optionally, one or more effervescent agents can be used as disintegrants and/or to enhance organoleptic properties of pharmaceutical compositions of the invention. When present in pharmaceutical compositions of the invention to promote dosage form disintegration, one or more effervescent agents are preferably present in a total amount of about 30% to about 75%, and preferably about 45% to about 70%, for example about 60%, by weight of the pharmaceutical composition.

According to a particularly preferred embodiment of the invention, an effervescent agent, present in a solid dosage form in an amount less than that effective to promote disintegration of the dosage form, provides improved dispersion of the zafirlukast in an aqueous medium. Without being bound by theory, it is believed that the effervescent agent is effective to accelerate dispersion of zafirlukast from the dosage form in the gastrointestinal tract, thereby further enhancing absorption and rapid onset of therapeutic effect. When present in a pharmaceutical composition of the invention to promote intragastrointestinal dispersion but not to enhance disintegration, an effervescent agent is preferably present in an amount of about 1% to about 20%, more preferably about 2.5% to about 15%, and still more preferably about 5% to about 10%, by weight of the pharmaceutical composition.

An “effervescent agent” herein is an agent comprising one or more compounds which, acting together or individually, evolve a gas on contact with water. The gas evolved is generally oxygen or, most commonly, carbon dioxide. Preferred effervescent agents comprise an acid and a base that react in the presence of water to generate carbon dioxide gas. Preferably, the base comprises an alkali metal or alkaline earth metal carbonate or bicarbonate and the acid comprises an aliphatic carboxylic acid. Non-limiting examples of suitable bases as components of effervescent agents useful in the invention include carbonate salts (e.g., calcium carbonate), bicarbonate salts (e.g., sodium bicarbonate), sesquicarbonate salts, and mixtures thereof. Calcium carbonate is a preferred base.

Non-limiting examples of suitable acids as components of effervescent agents useful in the invention include citric acid, tartaric acid (as D-, L-, or D/L-tartaric acid), malic acid, maleic acid, fumaric acid, adipic acid, succinic acid, acid anhydrides of such acids, acid salts of such acids, and mixtures thereof. Citric acid is a preferred acid.

In a preferred embodiment of the invention, where the effervescent agent comprises an acid and a base, the weight ratio of the acid to the base is about 1:100 to about 100:1, more preferably about 1:50 to about 50:1, and still more preferably about 1:10 to about 10:1. In a further preferred embodiment of the invention, where the effervescent agent comprises an acid and a base, the ratio of the acid to the base is approximately stoichiometric.

Solid dosage forms of the invention can be prepared by any suitable process, not limited to processes described herein.

An illustrative process comprises (a) a step of blending a zafirlukast salt of the invention with one or more excipients to form a blend, and (b) a step of tableting or encapsulating the blend to form tablets or capsules, respectively. When forming a tablet, such a process is typically called direct compression tableting. When preparing a capsule, the process is typically called a direct fill procedure.

In another process, solid dosage forms are prepared by a process comprising (a) a step of blending a zafirlukast salt of the invention with one or more excipients to form a blend, (b) a step of granulating the blend to form a granulate, and (c) a step of tableting or encapsulating the blend to form tablets or capsules respectively. Step (b) can be accomplished by any dry or wet granulation technique known in the art, but is preferably a dry granulation step. A zafirlukast salt of the present invention is advantageously granulated to form particles of about 1 micron to about 100 microns, about 5 microns to about 50 microns, or about 10 microns to about 25 microns. One or more diluents, one or more disintegrants and one or more binding agents are preferably added, for example in the blending step, a wetting agent can optionally be added, for example in the granulating step, and one or more disintegrants are preferably added after granulating but before tableting or encapsulating. A lubricant is preferably added before tableting. Blending and granulating can be performed independently under low or high shear. A process is preferably selected that forms a granulate that is uniform in drug content, that readily disintegrates, that flows with sufficient ease so that weight variation can be reliably controlled during capsule filling or tableting, and that is dense enough in bulk so that a batch can be processed in the selected equipment and individual doses fit into the specified capsules or tablet dies.

In addition, zafirlukast can be prepared as an oral fast-melt formulation or a rapidly-disintegrating oral formulation, where the process of preparing such formulations is described in U.S. Publication Nos. 2002/0119193 and 2002/0071857, the contents of which are incorporated herein by reference.

In an alternative embodiment, solid dosage forms are prepared by a process that includes a spray drying step, wherein a zafirlukast salt is suspended with one or more excipients in one or more sprayable liquids, preferably a non-protic (e.g., non-aqueous or non-alcoholic) sprayable liquid, and then is rapidly spray dried over a current of warm air.

A granulate or spray dried powder resulting from any of the above illustrative processes can be compressed or molded to prepare tablets or encapsulated to prepare capsules. Conventional tableting and encapsulation techniques known in the art can be employed. Where coated tablets are desired, conventional coating techniques are suitable.

Zafirlukast dosage forms of the invention preferably comprise zafirlukast in a daily dosage amount of about 2 mg to about 80 mg, more preferably about 5 mg to about 40 mg, such as about 5 mg, about 10 mg, about 20 mg or about 40 mg.

Zafirlukast salts of the invention can be administered to a subject orally, parenterally (e.g., as an intravenous, intramuscular, intraperitoneal or subcutaneous injection), topically, intranasally, by aerosol or rectally. The form in which the zafirlukast salt is administered, for example, powder, tablet, capsule, solution, or emulsion, depends in part on the route by which it is administered. Preferred routes of administration are orally or via an injection.

Pharmaceutical compositions of the invention comprise one or more orally deliverable dose units. Each dose unit comprises zafirlukast in a therapeutically effective amount that is preferably about 2 mg to about 80 mg. The term “dose unit” herein means a portion of a pharmaceutical composition that contains an amount of a therapeutic or prophylactic agent, in the present case zafirlukast, suitable for a single oral administration to provide a therapeutic effect. Typically one dose unit, or a small plurality (up to about 4) of dose units, in a single administration provides a dose comprising a sufficient amount of the agent to result in the desired effect. Administration of such doses can be repeated as required, typically at a dosage frequency of 1 to about 4 times per day, preferably twice daily.

It will be understood that a therapeutically effective amount of zafirlukast for a subject is dependent inter alia on the body weight of the subject. A “subject” to which a zafirlukast salt or a pharmaceutical composition thereof can be administered includes a human subject of either sex and of any age, and also includes any nonhuman animal, particularly a warm-blooded animal, more particularly a domestic or companion animal, illustratively a cat, dog or horse. When the subject is a child or a small animal (e.g., a dog), for example, an amount of zafirlukast (measured as the neutral form of zafirlukast, that is, not including counterions in a salt or water in a hydrate) relatively low in the preferred range of about 2 mg to about 80 mg is likely to provide blood serum concentrations consistent with therapeutic effectiveness. Where the subject is an adult human or a large animal (e.g., a horse), achievement of such blood serum concentrations of zafirlukast is likely to require dose units containing a relatively greater amount of zafirlukast.

Typical dose units in a pharmaceutical composition of the invention contain about 1, 2, 3, 5, 7.5, 10, 15, 20, 25, 30, 35 or 40 mg of zafirlukast. For an adult human, a therapeutically effective amount of zafirlukast per dose unit in a composition of the present invention is typically about 5 mg to about 20 mg. Especially preferred amounts of zafirlukast per dose unit are about 10 mg to about 20 mg, for example about 10 mg or about 20 mg.

A dose unit containing a particular amount of zafirlukast can be selected to accommodate any desired frequency of administration used to achieve a desired daily dosage. The daily dosage and frequency of administration, and therefore the selection of appropriate dose unit, depends on a variety of factors, including the age, weight, sex and medical condition of the subject, and the nature and severity of the condition or disorder, and thus may vary widely.

For pain management, pharmaceutical compositions of the present invention can be used to provide a daily dosage of zafirlukast of about 5 mg to about 80 mg, preferably about 20 mg to about 40 mg. The daily dose can be administered in one to about four doses per day. Administration at a rate of one 20 mg dose unit one or two times a day is preferred.

The term “oral administration” herein includes any form of delivery of a therapeutic agent or a composition thereof to a subject wherein the agent or composition is placed in the mouth of the subject, whether or not the agent or composition is immediately swallowed. Thus, “oral administration” includes buccal and sublingual as well as esophageal administration. Absorption of the agent can occur in any part or parts of the gastrointestinal tract including the mouth, esophagus, stomach, duodenum, ileum and colon. The term “orally deliverable” herein means suitable for oral administration.

Pharmaceutically acceptable salts of zafirlukast can be administered by controlled- or delayed-release means. Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, “Controlled Release Dosage Form Design”, pgs. 231-238, Technomic Publishing, Lancaster, Pa.: 2000).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under dosing a drug (i.e., going below the minimum therapeutic levels) as well as exceeding the toxicity level for the drug.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the zafirlukast salts and compositions of the invention. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of zafirlukast and thus effect controlled delivery of the drug. Examples of specific anion exchangers include, but are not limited to, Duolite® A568 and Duolite® AP143 (Rohm & Haas, Spring House, Pa. USA).

One embodiment of the invention encompasses a unit dosage form which comprises a pharmaceutically acceptable salt of zafirlukast (e.g., a potassium salt), or a polymorph, solvate, hydrate, dehydrate, co-crystal, anhydrous, or amorphous form thereof, and one or more pharmaceutically acceptable excipients or diluents, wherein the pharmaceutical composition or dosage form is formulated for controlled-release. Specific dosage forms utilize an osmotic drug delivery system.

A particular and well-known osmotic drug delivery system is referred to as OROS® (Alza Corporation, Mountain View, Calif. USA). This technology can readily be adapted for the delivery of compounds and compositions of the invention. Various aspects of the technology are disclosed in U.S. Pat. Nos. 6,375,978 B1; 6,368,626 B1; 6,342,249 B1; 6,333,050 B2; 6,287,295 B1; 6,283,953 B1; 6,270,787 B1; 6,245,357 B1; and 6,132,420; each of which is incorporated herein by reference. Specific adaptations of OROS® that can be used to administer compounds and compositions of the invention include, but are not limited to, the OROS® Push-Pull™, Delayed Push-Pull™, Multi-Layer Push-Pull™, and Push-Stick™ Systems, all of which are well known. See, e.g., http://www.alza.com. Additional OROS® systems that can be used for the controlled oral delivery of compounds and compositions of the invention include OROS®-CT and L-OROS®. Id.; see also, Delivery Times, vol. II, issue II (Alza Corporation).

Conventional OROS® oral dosage forms are made by compressing a drug powder (e.g., zafirlukast salt) into a hard tablet, coating the tablet with cellulose derivatives to form a semi-permeable membrane, and then drilling an orifice in the coating (e.g., with a laser). (Kim, Cherng-ju, “Controlled Release Dosage Form Design”, pgs. 231-238 Technomic Publishing, Lancaster, Pa.: 2000). The advantage of such dosage forms is that the delivery rate of the drug is not influenced by physiological or experimental conditions. Even a drug with a pH-dependent solubility can be delivered at a constant rate regardless of the pH of the delivery medium. But because these advantages are provided by a build-up of osmotic pressure within the dosage form after administration, conventional OROS® drug delivery systems cannot be used to effectively deliver drugs with low water solubility.

A specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a dry or substantially dry state drug layer located within the cavity adjacent to the exit orifice and in direct or indirect contacting relationship with the expandable layer; and a flow-promoting layer interposed between the inner surface of the wall and at least the external surface of the drug layer located within the cavity, wherein the drug layer comprises a salt of zafirlukast, or a polymorph, solvate, hydrate, dehydrate, co-crystal, anhydrous, or amorphous form thereof. See U.S. Pat. No. 6,368,626, the entirety of which is incorporated herein by reference.

Another specific dosage form of the invention comprises: a wall defining a cavity, the wall having an exit orifice formed or formable therein and at least a portion of the wall being semipermeable; an expandable layer located within the cavity remote from the exit orifice and in fluid communication with the semipermeable portion of the wall; a drug layer located within the cavity adjacent the exit orifice and in direct or indirect contacting relationship with the expandable layer; the drug layer comprising a liquid, active agent formulation absorbed in porous particles, the porous particles being adapted to resist compaction forces sufficient to form a compacted drug layer without significant exudation of the liquid, active agent formulation, the dosage form optionally having a placebo layer between the exit orifice and the drug layer, wherein the active agent formulation comprises a salt of zafirlukast, or a polymorph, solvate, hydrate, dehydrate, co-crystal, anhydrous, or amorphous form thereof. See U.S. Pat. No. 6,342,249, the entirety of which is incorporated herein by reference. As stated previously, zafirlukast possesses leukotriene antagonist properties. Thus, it antagonizes the actions of one or more of the arachidonic acid metabolites known as leukotrienes, for example, C₄, D₄ and/or E₄, which are known to be powerful spasmogens (particularly in the lung), to increase vascular permeability and have been implicated in the pathogenesis of asthma and inflammation (see J. L. Marx, Science, 1982, 215, 1380-1383) as well as of endotoxic shock (see J. A. Cook, et al., J. Pharmacol. Exp. Ther., 1985, 235, 470) and traumatic shock (see C. Denzlinger, et al., Science, 1985, 230, 330). Compositions of the present invention (e.g., zafirlukast potassium salt, zafirlukast methanol solvate) are thus useful in the treatment of diseases in which leukotrienes are implicated and in which antagonism of their action is desired. Such diseases include, for example, allergic pulmonary disorders such as asthma, hay fever and allergic rhinitis and certain inflammatory diseases such as bronchitis, ectopic and atopic eczema, psoriasis, as well as vasospastic cardiovascular disease, and endotoxic and traumatic shock conditions. The compounds and compositions of the present invention are particularly useful in the treatment of asthma.

### Exemplification

Below are standard procedures for acquiring Raman, PXRD, DSC and TGA data herein. These procedures will be followed for each respective method of analysis herein unless otherwise indicated.

Procedure for Raman Acquisition, Filtering and Binning

Acquisition

The sample was either left in the glass vial in which it was processed or an aliquot of the sample was transferred to a glass slide. The glass vial or slide was positioned in the sample chamber. The measurement was made using an Almega™ Dispersive Raman (Almega™ Dispersive Raman, Thermo-Nicolet, 5225 Verona Road, Madison, Wis. 53711-4495) system fitted with a 785 nm laser source. The sample was manually brought into focus using the microscope portion of the apparatus with a 10× power objective (unless otherwise noted), thus directing the laser onto the surface of the sample. The spectrum was acquired using the parameters outlined in Table 1. (Exposure times and number of exposures may vary; changes to parameters will be indicated for each acquisition.)

Filtering and Binning

Each spectrum in a set was filtered using a matched filter of feature size 25 to remove background signals, including glass contributions and sample fluorescence. This is particularly important as large background signal or fluorescence limit the ability to accurately pick and assign peak positions in the subsequent steps of the binning process. Filtered spectra were binned using the peak pick and bin algorithm with the parameters given in Table 2. The sorted cluster diagrams for each sample set and the corresponding cluster assignments for each spectral file were used to identify groups of samples with similar spectra, which was used to identify samples for secondary analyses.

TABLE 1
Raman Spectral acquisition parameters

| Parameter | Setting Used |
|---|---|
| Exposure time (s) | 2.0 |
| Number of exposures | 10 |
| Laser source wavelength (nm) | 785 |
| Laser power (%) | 100 |
| Aperture shape | pin hole |
| Aperture size (um) | 100 |
| Spectral range (cm⁻¹) | 104-3428 |
| Grating position | Single |
| Temperature at acquisition | 24.0 |
| (degrees C.) | |

TABLE 2
Raman Filtering and Binning Parameters

| Parameter | Setting Used |
|---|---|
| Filtering Parameters | |
| Filter type | Matched |
| Filter size | 25 |
| QC Parameters | |
| Peak Height Threshold | 1000 |
| Region for noise test (cm⁻¹) | 0-10000 |
| RMS noise threshold | 10000 |
| Automatically eliminate | Yes |
| failed spectra | |
| Region of Interest | |
| Include (cm⁻¹) | 104-3428 |
| Exclude region I (cm⁻¹) | |
| Exclude region II (cm⁻¹) | |
| Exclude region III (cm⁻¹) | |
| Exclude region IV (cm⁻¹) | |
| Peak Pick Parameters | |
| Peak Pick Sensitivity | Variable |
| Peak Pick Threshold | 100 |
| Peak Comparison Parameters | |
| Peak Window (cm⁻¹) | 2 |
| Analysis Parameters | |
| Number of clusters | Variable |

Procedure for Powder X-Ray Diffraction (PXRD)

All powder x-ray diffraction patterns were obtained using the D/Max Rapid X-ray Diffractometer (D/Max Rapid, Contact Rigaku/MSC, 9009 New Trails Drive, The Woodlands, Tex., USA 77381-5209) equipped with a copper source (Cu/K_{α}1.5406 angstroms), manual x-y stage, and 0.3 mm collimator. The sample was loaded into a 0.3 mm boron rich glass capillary tube (e.g., Charles Supper Company, 15 Tech Circle, Natick, Mass. 01760-1024) by sectioning off one end of the tube and tapping the open, sectioned end into a bed of the powdered sample or into the sediment of a slurried precipitate. Note, precipitate can be amorphous or crystalline. The loaded capillary was mounted in a holder that was secured into the x-y stage. A diffractogram was acquired (e.g., Control software: RINT Rapid Control Software, Rigaku Rapid/XRD, version 1.0.0, © 1999 Rigaku Co.) under ambient conditions at a power setting of 46 kV at 40 mA in reflection mode, while oscillating about the omega-axis from 0-5 degrees at 1 degree/s and spinning about the phi-axis at 2 degrees/s. The exposure time was 15 minutes unless otherwise specified. The diffractogram obtained was integrated over 2-theta from 2-60 degrees and chi (1 segment) from 0-360 degrees at a step size of 0.02 degrees using the cyllnt utility in the RINT Rapid display software (Analysis software: RINT Rapid display software, version 1.18, Rigaku/MSC.) provided by Rigaku with the instrument. The dark counts value was set to 8 as per the system calibration (System set-up and calibration by Rigaku); normalization was set to average; the omega offset was set to 180°; and no chi or phi offsets were used for the integration. The analysis software JADE XRD Pattern Processing, versions 5.0 and 6.0 (⁸1995-2002, Materials Data, Inc.) was also used.

The relative intensity of peaks in a diffractogram is not necessarily a limitation of the PXRD pattern because peak intensity can vary from sample to sample, e.g., due to crystalline impurities. Further, the angles of each peak can vary by about +/−0.1 degrees, preferably +/−0.05. The entire pattern or most of the pattern peaks may also shift by about +/−0.1 degree due to differences in calibration, settings, and other variations from instrument to instrument and from operator to operator.

Procedure for Differential Scanning Calorimetry (DSC)

An aliquot of the sample was weighed into an aluminum sample pan. (e.g., Pan part # 900786.091; lid part # 900779.901; TA Instruments, 109 Lukens Drive, New Castle, Del. 19720) The sample pan was sealed either by crimping for dry samples or press fitting for wet samples (e.g., hydrated or solvated samples). The sample pan was loaded into the apparatus (DSC: Q1000 Differential Scanning Calorimeter, TA Instruments, 109 Lukens Drive, New Castle, Del. 19720), which is equipped with an autosampler, and a thermogram was obtained by individually heating the sample (e.g., Control software: Advantage for QW—Series, version 1.0.0.78, Thermal Advantage Release 2.0, ©2001 TA instruments—Water LLC) at a rate of 10 degrees C./min from Tₘᵢₙ (typically 20 degrees C.) to Tₘₐₓ (typically 300 degrees C.) (Heating rate and temperature range may vary, changes to these parameters will be indicated for each sample) using an empty aluminum pan as a reference. Dry nitrogen (e.g., Compressed nitrogen, grade 4.8, BOC Gases, 575 Mountain Avenue, Murray Hill, N.J. 07974-2082) was used as a sample purge gas and was set at a flow rate of 50 mL/min. Thermal transitions were viewed and analyzed using the analysis software (Analysis Software: Universal Analysis 2000 for Windows 95/95/2000/NT, version 3.1E; Build 3.1.0.40, © 1991-2001TA instruments— Water LLC) provided with the instrument.

Procedure for Thermogravimetric Analysis (TGA)

An aliquot of the sample was transferred into a platinum sample pan. (Pan part # 952019.906; TA Instruments, 109 Lukens Drive, New Castle, Del. 19720) The pan was placed on the loading platform and was then automatically loaded into the apparatus (TGA: Q500 Thermogravimetric Analyzer, TA Instruments, 109 Lukens Drive, New Castle, Del. 19720) using the control software (Control software: Advantage for QW-Series, version 1.0.0.78, Thermal Advantage Release 2.0, © 2001 TA instruments—Water LLC). Thermograms were obtained by individually heating the sample at 10 degrees C. /min from 25 degrees C. to 300 degrees C. (Heating rate and temperature range may vary, changes in parameters will be indicated for each sample) under flowing dry nitrogen (e.g., Compressed nitrogen, grade 4.8, BOC Gases, 575 Mountain Avenue, Murray Hill, N.J. 07974-2082), with a sample purge flow rate of 60 mL/min and a balance purge flow rate of 40 mL/min. Thermal transitions (e.g. weight changes) were viewed and analyzed using the analysis software (Analysis Software: Universal Analysis 2000 for Windows 95/95/2000/NT, version 3.1E; Build 3.1.0.40, © 1991-2001TA instruments—Water LLC) provided with the instrument.

For PXRD data herein, including Figures and written description, each composition of the present invention may be characterized by any one, any two, any three, any four, any five, any six, any seven, any eight or more of the 2 theta angle peaks. Any one, two, three, four, five, or six DSC transitions can also be used to characterize the compositions of the present invention. Any one, two, three, four, five, or six or more Raman scattering peaks can also be used to characterize the compositions of the present invention. The different combinations of the PXRD peaks, Raman peaks, and the DSC transitions can also be used to characterize the compositions.

### Example 1

Preparation and Characterization of a Potassium Salt of Zafirlukast

Zafirlukast was isolated from ACCOLATE® tablets. The tablets were crushed and suspended in tetrahydrofuran (THF). The solution was collected via filtration and then following evaporation of the solvent, an oil was obtained.

A solution of zafirlukast potassium salt was prepared by adding potassium tert-butoxide (1.0 M in THF; 0.30 mL; 0.30 mmol) to a suspension of zafirlukast (156 mg; 0.272 mmol) in methanol (8.0 mL). An aliquot (1.05 mL, 20.5 mg zafirlukast potassium salt) was removed and evaporated to an oil in a separate vial. To the oil was added toluene (2.0 mL) followed by 2-butanol (0.2 mL). Crystals formed within minutes and were allowed to sit overnight with slow evaporation of the solvent. The solid was then collected and isolated via filtration. Amorphous solid was obtained from crystallization attempts in pure toluene. Crystals were obtained with various toluene/2-butanol mixtures as well as toluene/1-butanol and toluene/isopropanol mixtures.

Another preparation was also utilized for the synthesis of zafirlukast potassium salt. To a suspension of zafirlukast (150 mg; 0.260 mmol) in methanol (3.0 mL) was added a solution of potassium hydroxide (15.6 mg; 0.278 mmol) in methanol (4.0 mL). The mixture was heated to 50 degrees C. and sonicated to dissolve the drug. The solution was then filtered through a 0.2 micrometer PTFE syringe filter and was allowed to sit overnight after a seed crystal was added. Some solid had crystallized overnight and the mixture was cooled to 0 degrees C. for 20 minutes to further crystallize the remaining drug. The solid was collected via filtration and washed with cold methanol (5.0 mL). The solid was then suspended in water and filtered. The solid was dried with flowing nitrogen gas for 1 hour and collected.

The potassium salt of zafirlukast does not convert back to the neutral form under aqueous conditions, either neutral or acidic. The PXRD diffractogram remains unchanged after exposure to these conditions.

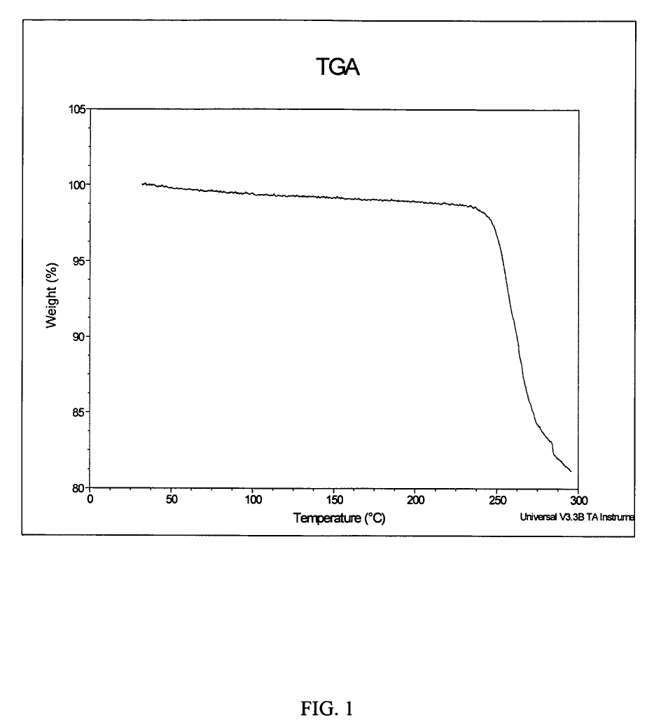

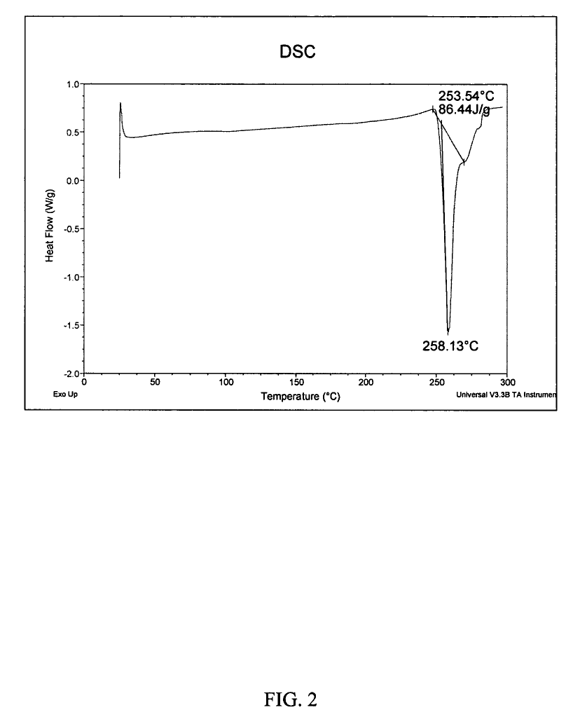

Zafirlukast potassium salt was characterized by thermogravimetric analysis (TGA), differential scanning calorimetry (DSC), powder x-ray diffraction (PXRD), and elemental analysis. TGA analysis showed the zafirlukast potassium salt loses about 2 percent to about 5 percent of its weight between room temperature and about 250 degrees C. (FIG. 1). The potassium salt is characterized by a sharp endothermic transition at about 258 degrees C. (FIG. 2). The PXRD analysis showed peaks occurring at, for example, 2 theta angles of 5.37, 7.77, 10.69, 12.49, 13.73, 15.03, 17.05, 19.59, 24.09, and 27.59 degrees. Any combination of one, two, three, four, five, six, seven, eight, nine, or more of the above PXRD peaks or those in FIG. 3 are characteristic of zafirlukast potassium salt. The results of elemental analysis can be found in Table I below.

TABLE I
Elemental Analysis Results of Zafirlukast Potassium Salt

| Element | Percent (Calculated) | Percent (Actual) |
|---|---|---|
| C | 60.66 | 60.59 |
| H | 5.26 | 5.30 |
| N | 6.85 | 6.77 |
| K | 6.37 | 6.35 |

### Example 2

Crystallization of Zafirlukast as the Methanol Solvate

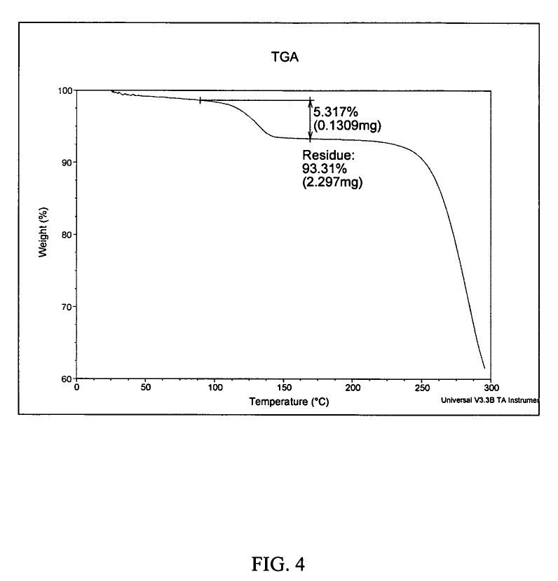

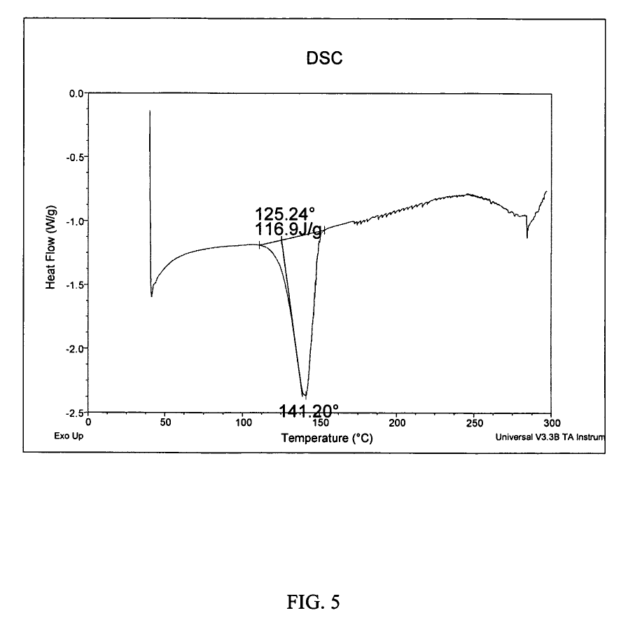

The methanol solvate was prepared via recrystallization of the amorphous form from methanol followed by cold filtration. Zafirlukast methanol solvate was characterized by thermogravimetric analysis (TGA), differential scanning calorimetry (DSC), powder x-ray diffraction (PXRD), and Raman spectroscopy. The TGA showed the zafirlukast methanol solvate loses about 5.3 percent of its weight between about 75 degrees C. and about 160 degrees C. (FIG. 4). The methanol solvate is characterized by an endothermic transition at about 141 degrees C. (FIG. 5). The PXRD analysis showed peaks occurring at, for example, 2 theta angles of 9.59, 10.69, 13.23, 15.45, 17.49, 18.05, 21.63, 22.69, and 26.83 degrees. Any combination of one, two, three, four, five, six, seven, eight, or more of the above PXRD peaks or those in FIG. 6 are characteristic of zafirlukast methanol solvate. The Raman spectrum showed scattering peaks at 1669, 1602, 1540, 1385, 1270, 1166, 776, and 592 cm⁻¹. Any combination of one, two, three, four, five, six, seven, eight, or more of the above Raman peaks or those in FIG. 7 are characteristic of zafirlukast methanol solvate.

While this invention has been particularly shown and described with references to preferred embodiments thereof, it will be understood by those skilled in the art that various changes in form and details may be made therein without departing from the scope of the invention encompassed by the appended claims.

1. A composition comprising a crystalline alkali metal salt of zafirlukast.

2. The composition of claim 1, further comprising a second crystalline entity.

3. The composition of claim 2, wherein the salt of zafirlukast and the second crystalline entity are co-crystallized.

4. The composition of claim 3, wherein the salt of zafirlukast and the second crystalline entity are co-crystallized as a solvated co-crystal, hydrated co-crystal, desolvated co-crystal or dehydrated co-crystal.

5. The composition of claim 1, wherein said salt is a potassium salt.

6. The composition of claim 5, wherein said salt is characterized by an endothermic transition at about 258 degrees C.

7. The composition of claim 5, wherein said salt loses about 2 percent to about 5 percent of its weight when the temperature is raised from room temperature to about 250 degrees C.

8. The composition of claim 4, wherein said composition comprises solvate molecules.

9. The composition of claim 8, wherein the solvate molecules are water.

10. The composition of claim 9, wherein said salt is greater than about 80% pure.

11. The composition of claim 9, wherein said salt is greater than about 90% pure.

12. The composition of claim 8, wherein the solvate molecules are alcohols.

13. The composition of claim 12, wherein the alcohol is selected from the group consisting of methanol, ethanol, n-propanol, and isopropanol.

14. A composition comprising a polymorph of the salt of claim 1.

15. A composition comprising a polymorph of the salt of claim 5.

16. The composition of claim 1, wherein said salt is partially or completely desolvated.

17. The composition of claim 1, wherein said salt is partially or completely dehydrated.

18. The composition of claim 5, wherein said salt is characterized by a powder X-ray diffraction pattern comprising peaks expressed in terms of 2-theta angles, wherein:
(a) said X-ray diffraction pattern comprises peaks at 5.37, 7.77, and 17.05 degrees;
(b) said X-ray diffraction pattern comprises peaks at 5.37, 10.69, and 15.03 degrees;
(c) said X-ray diffraction pattern comprises peaks at 17.05, 19.59, and 24.09 degrees;
(d) said X-ray diffraction pattern comprises peaks at 5.37, 7.77, 10.69, 15.03, 24.09, and 27.59 degrees;
(e) said X-ray diffraction pattern comprises peaks at 5.37, 12.49, 15.03, 17.05, 19.59, and 24.09 degrees;
(f) said X-ray diffraction pattern comprises peaks at 5.37 and 7.77 degrees;
(g) said X-ray diffraction pattern comprises peaks at 12.49 and 17.05 degrees;
(h) said X-ray diffraction pattern comprises a peak at 5.37 degrees; or
(i) said X-ray diffraction pattern comprises a peak at 10.69 degrees.

19. A method of preparing a crystalline salt of zafirlukast, which comprises:
(a) contacting zafirlukast with a solvent;
(b) reacting zafirlukast with at least one equivalent of one or more bases; and
(c) isolating the crystalline salt, thereby obtaining crystals of the salt of zafirlukast.

20. The method of claim 19, wherein:
(a) the solvent is an alcohol;
(b) the solvent is methanol;
(c) the base is a metal hydroxide or an alkoxide;
(d) the base is potassium hydroxide or potassium tert-butoxide;
(e) a seed crystal is added after step (b);
(f) the crystalline salt is isolated via filtration or evaporation of solvent; or
(g) the crystalline salt is isolated via removing the solvent followed by the addition of a solvent comprising toluene and an alcohol which is allowed to evaporate.

21. A pharmaceutical composition comprising a salt of zafirlukast.

22. The composition of claim 21, wherein said salt is potassium.

23. A method of treating a subject suffering from asthma comprising administering to said subject the composition of claim 1.

24. The method of claim 23, wherein the composition is administered to the subject orally.

25. The method of claim 24, wherein the composition is used in combination with other therapies.
